(19) United States
(12) Patent Application Publication
(10) Pub. No.: US 2018/0154048 A1
(43) Pub. Date: Jun. 7, 2018

(54) TUNABLE POROUS 3D BIODEGRADABLE, BIOCOMPATIBLE POLYMER/NANOMATERIAL SCAFFOLDS, AND FABRICATING METHODS AND APPLICATIONS OF SAME

(71) Applicant: Board Of Trustees Of The University Of Arkansas, Little Rock, AR (US)

(72) Inventors: Karrer M. Alghazali, Little Rock, AR (US); Zeid A. Nima, Little Rock, AR (US); Shawn E. Bourdo, Little Rock, AR (US); Alexandru S. Biris, Little Rock, AR (US)

(21) Appl. No.: 15/834,699

(22) Filed: Dec. 7, 2017

Related U.S. Application Data
(63) Continuation-in-part of application No. 15/624,425, filed on Jun. 15, 2017.
(60) Provisional application No. 62/431,076, filed on Dec. 7, 2016.

(51) Int. Cl. A61L 27/56; A61L 27/58; A61L 27/28; A61L 27/04; A61L 27/08; A61L 27/12; A61L 27/36; A61L 27/10; A61L 27/54; A61L 27/16; A61L 27/18; A61L 27/26; B33Y 80/00; B33Y 70/00; B29C 64/165; B33Y 10/00; B29C 64/379; B29C 59/14

(52) U.S. Cl. CPC A61L 27/56; B29L 2031/7532; A61L 27/28; A61L 27/047; A61L 27/08; A61L 27/12; A61L 27/3608; A61L 27/10; A61L 27/54; A61L 27/16; A61L 27/18; A61L 27/26; B33Y 80/00; B33Y 70/00; B29C 64/165; B33Y 10/00; B29C 64/379; B29C 59/142; A61L 2300/414; A61L 2300/412; A61L 2420/02; A61L 2300/604; A61L 2300/606; A61L 2400/12; A61L 2430/02; A61L 27/58

(57) ABSTRACT

The disclosure relates to a scaffold useable for tissue regeneration and methods for fabricating the scaffold. The scaffold includes a three-dimensional structure having a tunable porosity with interconnected channels and pores along with adjustable dimensions, and being formed of at least one of a first medium, a second medium, a third medium and a fourth medium. The first medium comprises one or more polymers that are biocompatible and biodegradable. The second medium comprises one or more soluble materials, and is mixable with the first medium. The third medium comprises fillers of one or more insoluble materials having structures with dimensions between 1 nm to 5 mm, and is mixable in a bulk or surface of the first medium or the second medium individually, or in a bulk or surface of a combination of the first and second media. The fourth medium comprises an agent.

### CROSS-REFERENCE TO RELATED PATENT APPLICATIONS

This application claims priority to and the benefit of, pursuant to 35 U.S.C. § 119(e), U.S. provisional patent application Ser. No. 62/431,076, filed Dec. 7, 2016, entitled “METHODS OF FABRICATING TUNABLE POROUS 3D BIODEGRADABLE, BIOCOMPATIBLE POLYMER/NANOMATERIAL SCAFFOLDS AND APPLICATIONS OF SAME”, by Karrer Alghazali et al., which is incorporated herein by reference in its entirety.

This application is also a continuation-in-part of U.S. patent application Ser. No. 15/624,425, filed Jun. 15, 2017, entitled “BONE REGENERATION USING BIODEGRADABLE POLYMERIC NANOCOMPOSITE MATERIALS AND APPLICATIONS OF THE SAME”, by Alexandru S. Biris, which is incorporated herein by reference in its entirety.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference is individually incorporated by reference. In terms of notation, hereinafter, [n] represents the nth reference cited in the reference list. For example, [1] represents the first reference cited in the reference list, namely, ALGHAZALI, K. M., NIMA, Z. A., HAMZAH, R. N., DHAR, M. S., ANDERSON, D. E. and BIRIS, A. S. 2015. Bone-tissue engineering: complex tunable structural and biological responses to injury, drug delivery, and cell-based therapies. Drug Metabolism Reviews, 47, 431-454.

### STATEMENT AS TO RIGHTS UNDER FEDERALLY-SPONSORED RESEARCH

This invention was made with government support under Contract No. W81XWH-15-1-0666 awarded by the Department of Defense (DOD-MRMCC). The government has certain rights in the invention.

### FIELD

The present disclosure relates generally to a biocompatible structure having one or more base structures for bone and tissue regeneration, and more particularly to methods of fabricating tunable porous three-dimension (3D) biodegradable, biocompatible polymer/nanomaterial scaffolds and applications of the same.

### BACKGROUND

The background description provided herein is for the purpose of generally presenting the context of the present disclosure. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the present disclosure.

Regenerative medicine devices have proven to be valuable for tissue regenerations [5], where traditional clinical products such as autografts, allografts, and xenografts have a lot of obstacles that might cause failures [1]. The necessity to create alternative regeneration treatments to reach clinical trials has brought noticeable developments to artificial regenerative medicine device designs [3]. Although most of these developments are successful, they all have problems and limitations.

Therefore, a heretofore unaddressed need exists in the art to address the aforementioned deficiencies and inadequacies.

### SUMMARY

One of the objectives of this disclosure is to provide a scaffold that is a multistructural composite with tunable characteristics for tissue regeneration as well as for delivery of bio-active molecules such as drugs, growth factors, and so on, and a fabricating method of the same.

In one aspect, the disclosure relates to a scaffold useable for tissue regeneration. In one embodiment, the scaffold includes a three-dimensional (3D) structure having a tunable porosity with interconnected channels and pores along with adjustable dimensions, and being formed of at least one of a first medium, a second medium, a third medium and a fourth medium. The first medium comprises one or more polymers that are biocompatible and biodegradable. The second medium comprises one or more soluble materials, and is mixable with the first medium. The third medium comprises fillers of one or more insoluble materials having structures with dimensions between 1 nm to 5 mm, and is mixable in a bulk or surface of the first medium or the second medium individually, or in a bulk or surface of a combination of the first and second media. The fourth medium comprises an agent.

In one embodiment, the 3D structure is capable of incubating or incorporating various types of nanoparticles, cells, bioactive materials, growth factors, and/or tissue regeneration enhancing drugs therein.

In one embodiment, internal and external surfaces of the 3D structure and/or a bulk of the 3D structure are coated with nanostructural materials.

In one embodiment, the 3D structure has a shape and size conforming to a shape and size of corresponding tissue that needs to be regenerated.

In one embodiment, a mixture of the first, second and third media is obtained in bulks, layers, or concentrically arranged geometries by using at least one process of mixing, spraying, electrospraying, extrusion, layer-by-layer deposition, and the likes.

In one embodiment, the mixture of the first, second and third media is operably exposed to the fourth medium to remove the second medium without adversely affecting the first and third media, so as to form a first composite.

In one embodiment, the fourth medium is operably removed from the first composite by at least one process of evaporating, drying, heating, vacuum drying, freeze-drying, and the likes, so as to form a second composite.

In one embodiment, the second composite is operably exposed to a plasma treatment for the surface modification to alter its surface chemistry, wherein the plasma treatment is performed in at least one gas of oxygen, nitrogen, helium, argon, and the likes.

In one embodiment, a concentration of the third medium is between 0 to 99.99% of the first medium in the second composite.

In one embodiment, the tunable porosity of the 3D structure is tunable with pore sizes from 0.1 nm to 10 mm, and wherein the surface area of the 3D structure is between 0.001 and 5000 m²/g.

In one embodiment, the tunable porosity is achievable through 3D printing.

In one embodiment, the one or more polymers comprise polyurethanes, polylactide (PLA), polyglycolide (PGA), poly(lactide-co-glycolide) (PLGA), poly(e-caprolactone), polydioxanone, polyanhydride, trimethylene carbonate, poly(β-hydroxybutyrate), poly(g-ethyl glutamate), poly(desaminotyrosinetyrosylhexyl ester iminocarbonate) (poly(DTH iminocarbonate)), poly(bisphenol A iminocarbonate), poly(ortho ester), polycyanoacrylate, polyphosphazene, a polymer derived from natural source including polysaccharides, proteins, or a mixture thereof.

In one embodiment, the first medium is combinable with ethanol, methanol, or other organic solvents or mixtures thereof.

In one embodiment, the one or more soluble materials (second medium) have a rate of degradation or dissolution that is faster than that of the first medium in a solvent, and comprise soluble crystals including sodium chloride, sugar, or other material.

In one embodiment, the one or more insoluble materials (third medium) comprise at least one or any combination of the following: (1) metal materials including gold, silver, copper, or other metals, or a combination of them, with micro-sized and/or nano-sized structures of various shapes including spheres, rods, platelets, cylinders, cubes, pyramids, cavities, nanoshells, nanocages, or the likes; (2) carbonaceous materials including nanotubes, graphene, nanofibers, nanoonions, nanocones, or the likes; (3) micro-sized or nano-sized hydroxyapatite; (4) bone component particles, and/or bone component nanoparticles; (5) calcium phosphate; (6) or micro-sized and/or micro-sized ceramics.

In one embodiment, the agent comprises deionized (DI) water, sodium hydroxide, ethanol, methanol, or other organic solvents, or mixtures thereof.

In another aspect, the disclosure relates to a method for fabricating a scaffold useable for tissue regeneration. In one embodiment, the method includes providing a first medium, a second medium, a third medium and a fourth medium, wherein the first medium comprises one or more polymers that are biocompatible and biodegradable; wherein the second medium comprises one or more soluble materials, and is mixable with the first medium; wherein the third medium comprises fillers of one or more insoluble materials having structures with dimensions between 1 nm to 5 mm, and is mixable in a bulk or surface of the first medium or the second medium individually, or in a bulk or surface of a combination of the first and second media; and wherein the fourth medium comprises an agent.

The method also includes forming a mixture of the first, second and third media in bulks, layers, or concentrically arranged geometries by at least one process of mixing, spraying, electrospraying, extrusion, layer-by-layer deposition, and the likes; exposing the mixture of the first, second and third media to the fourth medium to remove the second medium without adversely affecting the first and third media, so as to form a first composite; and removing the fourth medium from the first composite by at least one process of evaporating, drying, heating, vacuum drying, freeze-drying, and the likes, so as to form the scaffold. As formed, the scaffold comprises a three-dimensional (3D) structure having a tunable porosity with interconnected channels and pores along with adjustable dimensions.

In one embodiment, the method further includes performing a plasma treatment to the scaffold for the surface modification to alter its surface chemistry, wherein the plasma treatment is performed in at least one gas of oxygen, nitrogen, helium, argon, and the likes.

In one embodiment, the 3D structure is capable of incubating or incorporating various types of nanoparticles, cells, bioactive materials, growth factors, and/or tissue regeneration enhancing drugs therein.

In one embodiment, internal and external surfaces of the 3D structure and/or a bulk of the 3D structure are coated with nanostructural materials.

In one embodiment, the 3D structure has a shape and size conforming to a shape and size of corresponding tissue that needs to be regenerated.

In one embodiment, a concentration of the third medium is between 0 to 99.99% of the first medium in the scaffold.

In one embodiment, the tunable porosity of the 3D structure is tunable with pore sizes from 0.1 nm to 10 mm, and wherein the surface area of the 3D structure is between 0.001 and 5000 m²/g.

In one embodiment, the tunable porosity is achievable through 3D printing.

In one embodiment, the one or more polymers comprise polyurethanes, polylactide (PLA), polyglycolide (PGA), poly(lactide-co-glycolide) (PLGA), poly(e-caprolactone), polydioxanone, polyanhydride, trimethylene carbonate, poly(β-hydroxybutyrate), poly(g-ethyl glutamate), poly(desaminotyrosinetyrosylhexyl ester iminocarbonate) (poly(DTH iminocarbonate)), poly(bisphenol A iminocarbonate), poly(ortho ester), polycyanoacrylate, polyphosphazene, a polymer derived from natural source including polysaccharides, proteins, or a mixture thereof.

In one embodiment, the first medium is combinable with ethanol, methanol, or other organic solvents or mixtures thereof.

In one embodiment, the one or more soluble materials have a rate of degradation or dissolution that is faster than that of the first medium in a solvent, and comprise soluble crystals including sodium, chloride, sugar, or other material.

In one embodiment, the one or more insoluble materials comprise at least one of metal materials including gold, silver, copper, or other metals, or a combination of them, with micro-sized and/or nano-sized structures of various shapes including spheres, rods, platelets, cylinders, cubes, pyramids, cavities, nanoshells, nanocages, or the likes; carbonaceous materials including nanotubes, graphene, nanofibers, nanoonions, nanocones, or the likes; micro-sized or nano-sized hydroxyapatite; bone component particles, and/or bone component nanoparticles; calcium phosphate; and micro-sized and/or micro-sized ceramics.

In one embodiment, the agent comprises deionized (DI) water, sodium hydroxide, ethanol, methanol, or other organic solvents, or mixtures thereof.

In yet another aspect, the disclosure relates to a method for fabricating a scaffold useable for tissue regeneration. In one embodiment, the method includes providing a first medium, a second medium, a third medium and a fourth medium, wherein the first medium comprises one or more polymers that are biocompatible and biodegradable; wherein the second medium comprises one or more soluble materials, and is mixable with the first medium; wherein the third medium comprises fillers of one or more insoluble materials having structures with dimensions between 1 nm to 5 mm, and is mixable in a bulk or surface of the first medium or the second medium individually, or in a bulk or surface of a combination of the first and second media; and wherein the fourth medium comprises an agent.

The method also includes mixing the second medium with the first medium until a paste-like state is achieved, to form a mixture, wherein the mixing ratio between biodegradable polymer and the soluble crystals could be altered depend on the quantity of the porosity within the scaffold, in this mixture case around 90% porosity were achieved within the structure; exposing the mixture to the fourth medium to solidify the one or more polymers so as to form the scaffold; transferring the scaffold in a water bath that is placed on an orbital shaker and leaching the one or more soluble materials from the scaffold with DI water; and drying and sterilizing the scaffold.

In one embodiment, the mixing step comprises adding nanoparticles microparticles, growth factors, and/or tissue regeneration enhancing drugs when mixing the first and second media to form the mixture, so that the nanoparticles microparticles, and/or tissue regeneration enhancing drugs are incubated and incorporated within the scaffold.

In one embodiment, the method further comprises immersing the sterilized scaffold the inside the solution contain nanoparticles microparticles, growth factors, and/or tissue regeneration enhancing drugs for a predetermined period.

In one embodiment, the exposing step comprises placing the mixture in a syringe having desired size and diameter; and extruding the mixture by the syringe inside a container contains the fourth medium, so that the scaffold has a shape and size conforming to a shape and size of corresponding tissue that needs to be regenerated.

In one embodiment, the one or more polymers comprise polyurethanes, polylactide (PLA), polyglycolide (PGA), poly(lactide-co-glycolide) (PLGA), poly(e-caprolactone), polydioxanone, polyanhydride, trimethylene carbonate, poly(β-hydroxybutyrate), poly(g-ethyl glutamate), poly(desaminotyrosinetyrosylhexyl ester iminocarbonate) (poly(DTH iminocarbonate)), poly(bisphenol A iminocarbonate), poly(ortho ester), polycyanoacrylate, polyphosphazene, a polymer derived from natural source including polysaccharides, proteins, or a mixture thereof.

In one embodiment, the first medium is combinable with ethanol, methanol, or other organic solvents or mixtures thereof.

In one embodiment, the one or more soluble materials have a rate of degradation or dissolution that is faster than that of the first medium in a solvent, and comprise soluble crystals including sodium, chloride, sugar, or other material.

In one embodiment, the one or more insoluble materials comprise at least one of metal materials including gold, silver, copper, or other metals, or a combination of them, with micro-sized and/or nano-sized structures of various shapes including spheres, rods, platelets, cylinders, cubes, pyramids, cavities, nanoshells, nanocages, or the likes; carbonaceous materials including nanotubes, graphene, nanofibers, nanoonions, nanocones, or the likes; micro-sized or nano-sized hydroxyapatite; bone component particles, and/or bone component nanoparticles; calcium phosphate; and micro-sized and/or micro-sized ceramics.

In one embodiment, the agent comprises deionized (DI) water, sodium hydroxide, ethanol, methanol, or other organic solvents, or mixtures thereof.

In certain aspects, the disclosure relates to methods for fabrication of multistructural composite materials that support tissue regeneration or act as delivery devices for bio-active molecules. The multistructural composite has a tunable porosity, tunable mechanical properties, and architecture, and is defined such that it can support cellular proliferation, deliver various drugs of growth factors. The technology has the following functions: tissue regeneration, support cellular proliferation, deliver bio-active molecules.

These and other aspects of the invention will become apparent from the following description of the preferred embodiment taken in conjunction with the following drawings, although variations and modifications therein may be affected without departing from the spirit and scope of the novel concepts of the invention.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment.

FIGS. 1A-1D show SEM images of a 3D scaffold in different spots according to certain embodiments of the disclosure. The SEM images show the scaffold having a 3D structure having a tunable porosity with interconnected channels and pores along with adjustable dimensions.

FIGS. 2A and 2B show processes (steps) for fabricating a 3D scaffold according to certain embodiments of the disclosure.

### DETAILED DESCRIPTION

The disclosure will now be described more fully hereinafter with reference to the accompanying drawings, in which exemplary embodiments of the disclosure are shown. This invention may, however, be embodied in many different forms and should not be construed as limited to the embodiments set forth herein. Rather, these embodiments are provided so that this disclosure will be thorough and complete, and will fully convey the scope of the disclosure to those skilled in the art. Like reference numerals refer to like elements throughout.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the disclosure or of any exemplified term. Likewise, the disclosure is not limited to various embodiments given in this specification.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the disclosure.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” to another feature may have portions that overlap or underlie the adjacent feature.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top”, may be used herein to describe one element's relationship to another element as illustrated in the figures. It will be understood that relative terms are intended to encompass different orientations of the device in addition to the orientation shown in the figures. For example, if the device in one of the figures is turned over, elements described as being on the “lower” side of other elements would then be oriented on the “upper” sides of the other elements. The exemplary term “lower” can, therefore, encompass both an orientation of lower and upper, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, the terms “comprise” or “comprising”, “include” or “including”, “carry” or “carrying”, “has/have” or “having”, “contain” or “containing”, “involve” or “involving” and the like are to be understood to be open-ended, i.e., to mean including but not limited to.

As used herein, the phrase “at least one of A, B, and C” should be construed to mean a logical (A or B or C), using a non-exclusive logical OR. It should be understood that one or more steps within a method may be executed in different order (or concurrently) without altering the principles of the disclosure.

Typically, terms such as “about,” “approximately,” “generally,” “substantially,” and the like unless otherwise indicated mean within 20 percent, preferably within 10 percent, preferably within 5 percent, and even more preferably within 3 percent of a given value or range. Numerical quantities given herein are approximate, meaning that the term “about,” “approximately,” “generally,” or “substantially” can be inferred if not expressly stated.

Typically, “nanoscopic-scale,” “nanoscopic,” “nanometer-scale,” “nanoscale,” the “nano-” prefix, and the like refers to elements or articles having widths or diameters of less than about 1 μm, preferably less than about 100 nm in some cases. Specified widths can be smallest width (i.e. a width as specified where, at that location, the article can have a larger width in a different dimension), or largest width (i.e., where, at that location, the article's width is no wider than as specified, but can have a length that is greater), unless pointed out otherwise.

Embodiments of the invention are illustrated in detail hereinafter with reference to accompanying drawings. It should be understood that specific embodiments described herein are merely intended to explain the invention, but not intended to limit the invention. In accordance with the purposes of this invention, as embodied and broadly described herein, this invention, in certain aspects, relates to tunable porous three-dimension (3D) biodegradable, biocompatible polymer/nanomaterial scaffolds and fabricating methods and applications of the same.

In most of the tissue trauma, there is loss of more than one type of tissues in an implant surgical site of a human or an animal. A biocompatible structure can be adapted to include multiple base structures having different properties, thus facilitating regeneration of two or more tissues in the implant surgical site of the human or the animal, or facilitating regeneration of tissues in a non-implant surgical site of the human or the animal and then transferred to the implant site, or facilitating regeneration of tissues in vitro or in the lab and then transferred to the implant surgical site. Alternatively, the biocompatible structure can have only one base structure.

In certain aspects, the disclosure is to provide a scaffold that is a multistructural composite with tunable characteristics for tissue regeneration as well as for delivery of bio-active molecules such as drugs, growth factors, and so on, and a fabricating method of the same.

In one embodiment, the scaffold includes a 3D structure having a tunable porosity with interconnected channels and pores along with adjustable dimensions, and being formed of at least one of a first medium, a second medium, a third medium and a fourth medium.

The first medium comprises one or more polymers that are biocompatible and biodegradable. In one embodiment, the one or more polymers comprise polyurethanes, polylactide (PLA), polyglycolide (PGA), poly(lactide-co-glycolide) (PLGA), poly(e-caprolactone), polydioxanone, polyanhydride, trimethylene carbonate, poly(β-hydroxybutyrate), poly(g-ethyl glutamate), poly(desaminotyrosinetyrosylhexyl ester iminocarbonate) (poly(DTH iminocarbonate)), poly(bisphenol A iminocarbonate), poly(ortho ester), polycyanoacrylate, polyphosphazene, a polymer derived from natural source including polysaccharides, proteins, or a mixture thereof. In one embodiment, the first medium is combinable with ethanol, methanol, or other organic solvents or mixtures thereof.

The second medium comprises one or more soluble materials, and is mixable with the first medium. In one embodiment, the one or more soluble materials have a rate of degradation or dissolution that is faster than that of the first medium in a solvent, and comprise soluble crystals including sodium, chloride, sugar, or other material.

The third medium comprises fillers of one or more insoluble materials having structures with dimensions between 1 nm to 5 mm, and is mixable in a bulk or surface of the first medium or the second medium individually, or in a bulk or surface of a combination of the first and second media. In one embodiment, the one or more insoluble materials comprise at least one of metal materials including gold, silver, copper, or other metals, or a combination of them, with micro-sized and/or nano-sized structures of various shapes including spheres, rods, platelets, cylinders, cubes, pyramids, cavities, nanoshells, nanocages, or the likes; carbonaceous materials including nanotubes, graphene, nanofibers, nanoonions, nanocones, or the likes; micro-sized or nano-sized hydroxyapatite; bone component particles, and/or bone component nanoparticles; calcium phosphate; and micro-sized and/or micro-sized ceramics.

The fourth medium comprises an agent. In one embodiment, the agent comprises deionized (DI) water, sodium hydroxide, ethanol, methanol, or other organic solvents, or mixtures thereof.

In one embodiment, a mixture of the first, second and third media is obtained in bulks, layers, or concentrically arranged geometries by using at least one process of mixing, spraying, electrospraying, extrusion, layer-by-layer deposition, and the likes. In one embodiment, the mixture of the first, second and third media is operably exposed to the fourth medium to remove the second medium without adversely affecting the first and third media, so as to form a first composite.

In one embodiment, the fourth medium is operably removed from the first composite by at least one process of evaporating, drying, heating, vacuum drying, freeze-drying, and the likes, so as to form a second composite.

In one embodiment, the second composite is operably exposed to a plasma treatment for the surface modification to alter its surface chemistry, wherein the plasma treatment is performed in at least one gas of oxygen, nitrogen, helium, argon, and the likes.

In one embodiment, a concentration of the third medium is between 0 to 99.99% of the first medium in the second composite.

In one embodiment, the 3D structure has a shape and size conforming to a shape and size of corresponding tissue that needs to be regenerated.

In one embodiment, the 3D structure is capable of incubating or incorporating various types of nanoparticles, cells, bioactive materials, growth factors, and/or tissue regeneration enhancing drugs therein.

In one embodiment, internal and external surfaces of the 3D structure and/or a bulk of the 3D structure are coated with nanostructural materials.

In one embodiment, the tunable porosity of the 3D structure is tunable with pore sizes from 0.1 nm to 10 mm, and wherein the surface area of the 3D structure is between 0.001 and 5000 m²/g.

As formed, the artificial regenerative medicine scaffold is biocompatible, biodegradable and able to form any shape necessary based on the wound. The scaffold has a tunable porosity with interconnection channels, which is sufficient to allow cell migration, diffusion of the nutrition and bodily fluids [2, 4]. The scaffold incorporates within its structure or on its surface tissue regeneration enhancement additives, which are one or more of, but are not limited to:

cells, including, but are not limited to, epithelial cells, neurons, glial cells, astrocytes, podocytes, mammary epithelial cells, islet cells, endothelial cells, mesenchymal cells, stem cells, osteoblast, muscle cells, striated muscle cells, fibroblasts, hepatocytes, ligament fibroblasts, tendon fibroblasts, chondrocytes, or a mixture thereof;

bioactive materials, including, but are not limited to, proteins, enzymes, growth factors, amino acids, bone morphogenic proteins, platelet derived growth factors, vascular endothelial growth factors, or a mixture thereof;

drugs, antimicrobials, anti-inflammatory [6];

particles and nanoparticles, including, but are not limited to, gold, silver, copper, nanoparticles, nanorods, nanocubes, nanoplates, nanocavities, nanostars, nanopyramids, graphene, nanohydroxyapatite, hydroxyapatite, calcium phosphate, bone particles and nanoparticles, ceramic particles and nanoparticles, and so on; and

polymers and nanostructures and nano-sized polymers, biocompatible and biodegradable polymers, natural and synthetic polymers, hydrogels.

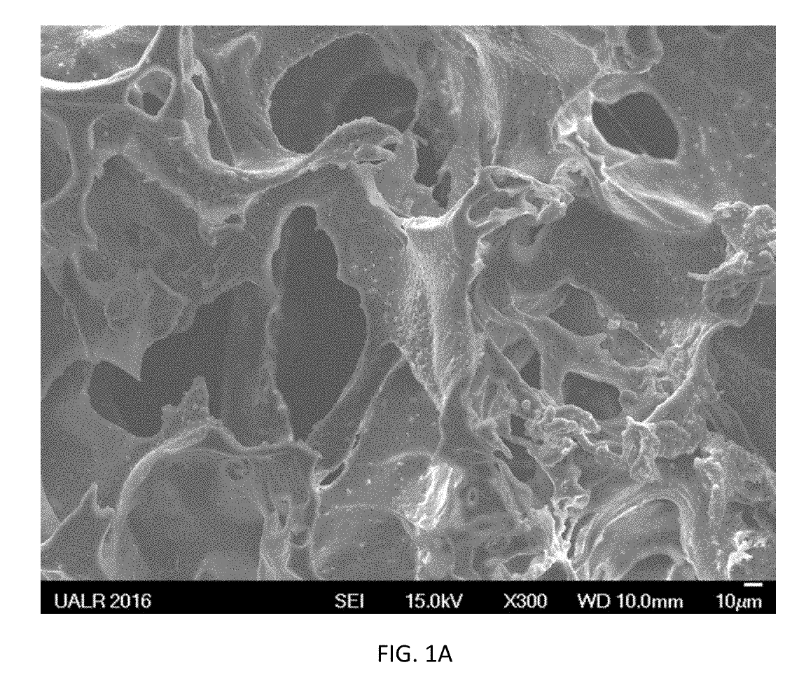

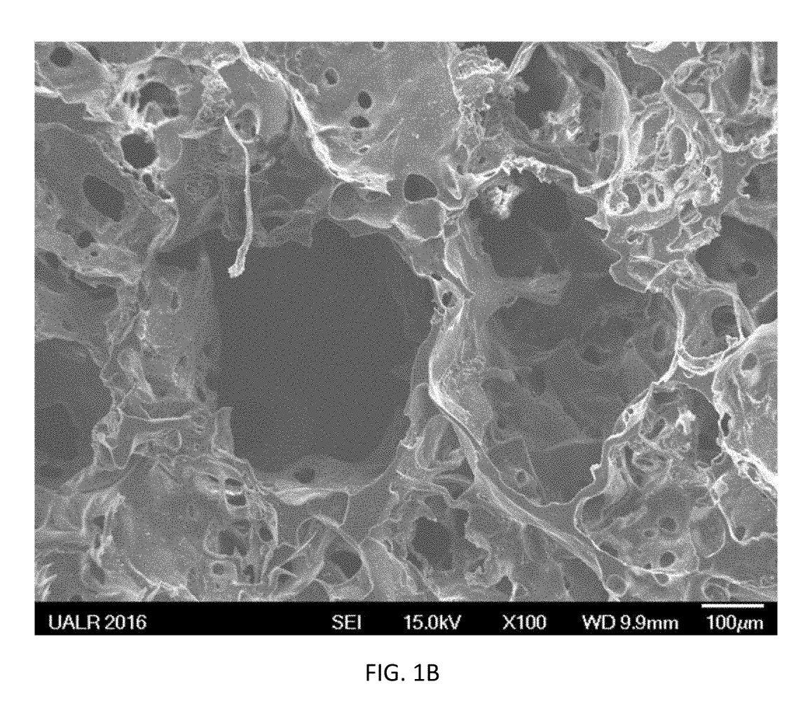

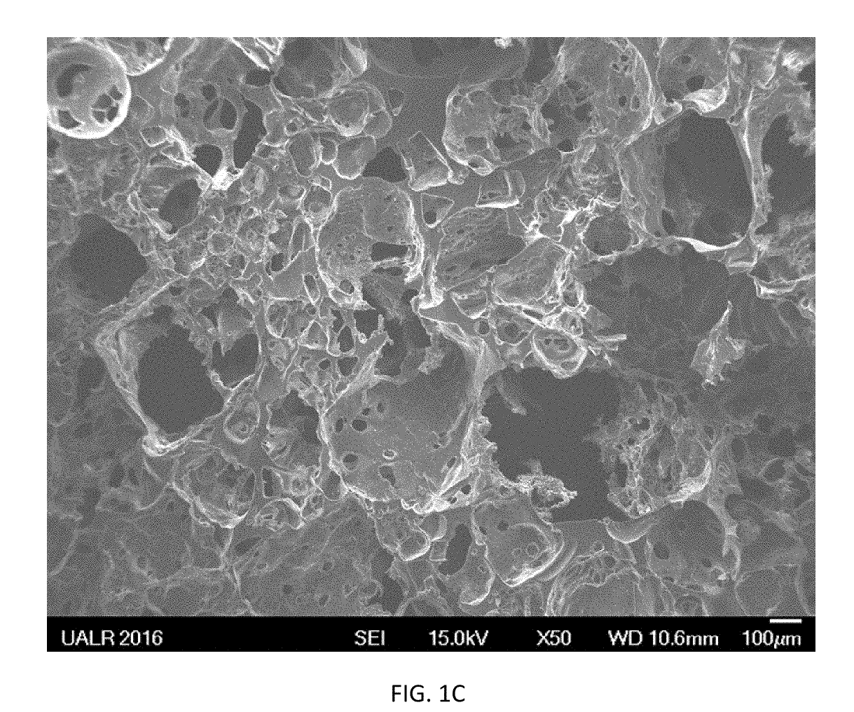

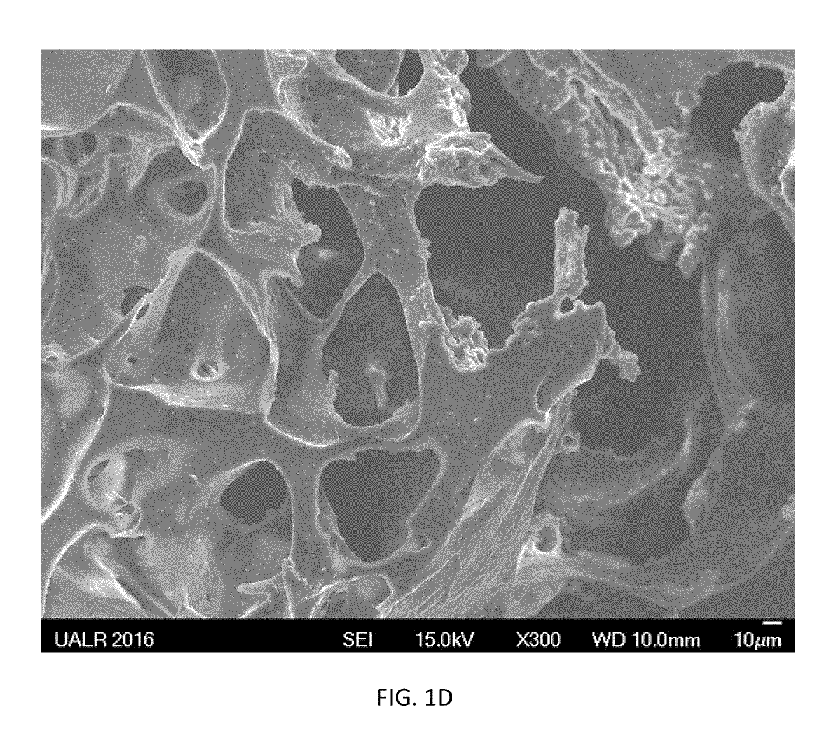

In addition, the 3D scaffold fits with different kinds of tissue regeneration such as nerve, bone, cartilage, arteries, skin, or any other type of hard/soft tissues where a scaffold is required for the regenerative processes. In certain embodiments, a tunable porosity with interconnected channels and pores along with adjustable dimensions for the scaffold is shown in FIGS. 1A-1D. In certain embodiments, the tunable porosity can be achieved through 3D printing. In addition, the ability to incubate or incorporate within the 3D structure of the scaffold with various types of nanoparticles, stem cells, tissue regeneration enhancing drugs is also unique. Furthermore, the scaffold composite can be arranged in layers with various materials in between.

In one aspect, the disclosure relates to a method for fabricating a scaffold useable for tissue regeneration. In one embodiment, the method includes providing a first medium, a second medium, a third medium and a fourth medium.

The first medium comprises one or more polymers that are biocompatible and biodegradable. In one embodiment, the one or more polymers comprise polyurethanes, polylactide (PLA), polyglycolide (PGA), poly(lactide-co-glycolide) (PLGA), poly(e-caprolactone), polydioxanone, polyanhydride, trimethylene carbonate, poly(β-hydroxybutyrate), poly(g-ethyl glutamate), poly(desaminotyrosinetyrosylhexyl ester iminocarbonate) (poly(DTH iminocarbonate)), poly(bisphenol A iminocarbonate), poly(ortho ester), polycyanoacrylate, polyphosphazene, a polymer derived from natural source including polysaccharides, proteins, or a mixture thereof. In one embodiment, the first medium is combinable with ethanol, methanol, or other organic solvents or mixtures thereof.

The second medium comprises one or more soluble materials, and is mixable with the first medium. In one embodiment, the one or more soluble materials have a rate of degradation or dissolution that is faster than that of the first medium in a solvent, and comprise soluble crystals including sodium, chloride, sugar, or other material.

The third medium comprises fillers of one or more insoluble materials having structures with dimensions between 1 nm to 5 mm, and is mixable in a bulk or surface of the first medium or the second medium individually, or in a bulk or surface of a combination of the first and second media. In one embodiment, the one or more insoluble materials comprise at least one of metal materials including gold, silver, copper, or other metals, or a combination of them, with micro-sized and/or nano-sized structures of various shapes including spheres, rods, platelets, cylinders, cubes, pyramids, cavities, nanoshells, nanocages, or the likes; carbonaceous materials including nanotubes, graphene, nanofibers, nanoonions, nanocones, or the likes; micro-sized or nano-sized hydroxyapatite; bone component particles, and/or bone component nanoparticles; calcium phosphate; and micro-sized and/or micro-sized ceramics.

The fourth medium comprises an agent. In one embodiment, the agent comprises deionized (DI) water, sodium hydroxide, ethanol, methanol, or other organic solvents, or mixtures thereof.

In addition, the method also includes forming a mixture of the first, second and third media in bulks, layers, or concentrically arranged geometries by at least one process of mixing, spraying, electrospraying, extrusion, layer-by-layer deposition, and the likes; exposing the mixture of the first, second and third media to the fourth medium to remove the second medium without adversely affecting the first and third media, so as to form a first composite; and removing the fourth medium from the first composite by at least one process of evaporating, drying, heating, vacuum drying, freeze-drying, and the likes, so as to form the scaffold. As formed, the scaffold comprises a three-dimensional (3D) structure having a tunable porosity with interconnected channels and pores along with adjustable dimensions.

In one embodiment, the method further includes performing a plasma treatment to the scaffold for the surface modification to alter its surface chemistry, wherein the plasma treatment is performed in at least one gas of oxygen, nitrogen, helium, argon, and the likes.

In one embodiment, the 3D structure is capable of incubating or incorporating various types of nanoparticles, cells, bioactive materials, growth factors, and/or tissue regeneration enhancing drugs therein.

In one embodiment, internal and external surfaces of the 3D structure and/or a bulk of the 3D structure are coated with nanostructural materials.

In one embodiment, the 3D structure has a shape and size conforming to a shape and size of corresponding tissue that needs to be regenerated.

In one embodiment, a concentration of the third medium is between 0 to 99.99% of the first medium in the scaffold.

In one embodiment, the tunable porosity of the 3D structure is tunable with pore sizes from 0.1 nm to 10 mm, and wherein the surface area of the 3D structure is between 0.001 and 5000 m²/g.

In one embodiment, the tunable porosity is achievable through 3D printing.

In another aspect, the disclosure relates to a method for fabricating a scaffold useable for tissue regeneration. In one embodiment, the method includes providing a first medium, a second medium, a third medium and a fourth medium.

The first medium comprises one or more polymers that are biocompatible and biodegradable. In one embodiment, the one or more polymers comprise polyurethanes, polylactide (PLA), polyglycolide (PGA), poly(lactide-co-glycolide) (PLGA), poly(e-caprolactone), polydioxanone, polyanhydride, trimethylene carbonate, poly(β-hydroxybutyrate), poly(g-ethyl glutamate), poly(desaminotyrosinetyrosylhexyl ester iminocarbonate) (poly(DTH iminocarbonate)), poly(bisphenol A iminocarbonate), poly(ortho ester), polycyanoacrylate, polyphosphazene, a polymer derived from natural source including polysaccharides, proteins, or a mixture thereof. In one embodiment, the first medium is combinable with ethanol, methanol, or other organic solvents or mixtures thereof.

The second medium comprises one or more soluble materials, and is mixable with the first medium. In one embodiment, the one or more soluble materials have a rate of degradation or dissolution that is faster than that of the first medium in a solvent, and comprise soluble crystals including sodium, chloride, sugar, or other material.

The third medium comprises fillers of one or more insoluble materials having structures with dimensions between 1 nm to 5 mm, and is mixable in a bulk or surface of the first medium or the second medium individually, or in a bulk or surface of a combination of the first and second media. In one embodiment, the one or more insoluble materials comprise at least one of metal materials including gold, silver, copper, or other metals, or a combination of them, with micro-sized and/or nano-sized structures of various shapes including spheres, rods, platelets, cylinders, cubes, pyramids, cavities, nanoshells, nanocages, or the likes; carbonaceous materials including nanotubes, graphene, nanofibers, nanoonions, nanocones, or the likes; micro-sized or nano-sized hydroxyapatite; bone component particles, and/or bone component nanoparticles; calcium phosphate; and micro-sized and/or micro-sized ceramics.

The fourth medium comprises an agent. In one embodiment, the agent comprises deionized (DI) water, sodium hydroxide, ethanol, methanol, or other organic solvents, or mixtures thereof.

The method also includes mixing the second medium with the first medium until a paste-like state is achieved, to form a mixture, wherein the mixing ratio between biodegradable polymer and the soluble crystals could be altered depend on the quantity of the porosity within the scaffold, in this mixture case around 90% porosity were achieved within the structure; exposing the mixture to the fourth medium to solidify the one or more polymers so as to form the scaffold; transferring the scaffold in a water bath that is placed on an orbital shaker and leaching the one or more soluble materials from the scaffold with DI water; and drying and sterilizing the scaffold.

In one embodiment, the mixing step comprises adding nanoparticles microparticles, growth factors, and/or tissue regeneration enhancing drugs when mixing the first and second media to form the mixture, so that the nanoparticles microparticles, and/or tissue regeneration enhancing drugs are incubated and incorporated within the scaffold.

In one embodiment, the method further comprises immersing the sterilized scaffold the inside the solution contain nanoparticles microparticles, growth factors, and/or tissue regeneration enhancing drugs for a predetermined period.

In one embodiment, the exposing step comprises placing the mixture in a syringe having desired size and diameter; and extruding the mixture by the syringe inside a container contains the fourth medium, so that the scaffold has a shape and size conforming to a shape and size of corresponding tissue that needs to be regenerated.

In certain aspects, the disclosure relates to methods for fabrication of multistructural composite materials that support tissue regeneration or act as delivery devices for bio-active molecules. The multistructural composite has a tunable porosity, tunable mechanical properties, and architecture, and is defined such that it can support cellular proliferation, deliver various drugs of growth factors. The technology has the following functions: tissue regeneration, support cellular proliferation, deliver bio-active molecules.

These and other aspects of the present invention are further described in the following section. Without intending to limit the scope of the invention, further exemplary implementations of the present invention according to the embodiments of the present invention are given below. Note that titles or subtitles may be used in the examples for the convenience of a reader, which in no way should limit the scope of the invention. Moreover, certain theories are proposed and disclosed herein; however, in no way should they, whether they are right or wrong, limit the scope of the invention so long as the invention is practiced according to the invention without regard for any particular theory or scheme of action.

The following is an exemplary embodiment according to the disclosure.

### Materials:

Medium A (or a first medium) includes a polymer or polymers combination of various ratios that are biocompatible, biodegradable, e.g., polyurethane, PMMA, PGLA, PLLA, etc., or other biodegradable polymers. Medium A can be combined with ethanol, methanol, or other organic solvents or mixtures thereof.

Medium B (or second medium) includes soluble crystals, for example, sodium chloride, and sugar or any other medium that preferentially dissolves in a solvent before medium A does, such as a fast degrading biocompatible, biodegradable polymer such as polyvinylpyrrolidone, poly(vinyl alcohol), poly(ethylene glycol), etc.

Sieves with different mesh size.

A tool to shape the composite in various shapes: cylindrical, films, tubular, spherical, triangular, conical, etc. For example: syringes and needle with different size and diameter, 3D printer that can produce various shapes and dimensions, spray-systems such gas-flow, electrospray, etc.

Drying environments that allow the solvent to be removed, either under ambient conditions or variable temperatures, pressures and electromagnetic excitations.

Beakers and flasks, mortar and pestle.

Medium C (or third medium) includes hard material fillers that include structures or combination of structures with dimensions between 1 nm to 5 mm and which could include: gold, silver, copper and other metals, or a combination of them (micro- and Nano-sized structures of various shapes-nanospheres, nanorods, nanoplates, cylindrical, nanocubes, nanopyramids, nanocavities), graphitic materials (nanotubes, graphene, nanofibers, nanoonions, nanocones, etc), hydroxyapatite (micro and nanosized), bone component particles and nanoparticles, calcium phosphate, ceramics of micro and nano sizes.

Medium D (or fourth medium) includes deionized (DI) water, sodium hydroxide, ethanol, methanol, or other organic solvents, or mixtures thereof.

Tissue regeneration enhancement drugs such as antimicrobials, anti-inflammatory, etc.

Growth factors, such as BMP, NGF, EGF, etc., proteins, DNA, SNA, extracellular matrix proteins.

Cells such as stem cells of various types, tissue specific cells, progenitors, etc.

### Method:

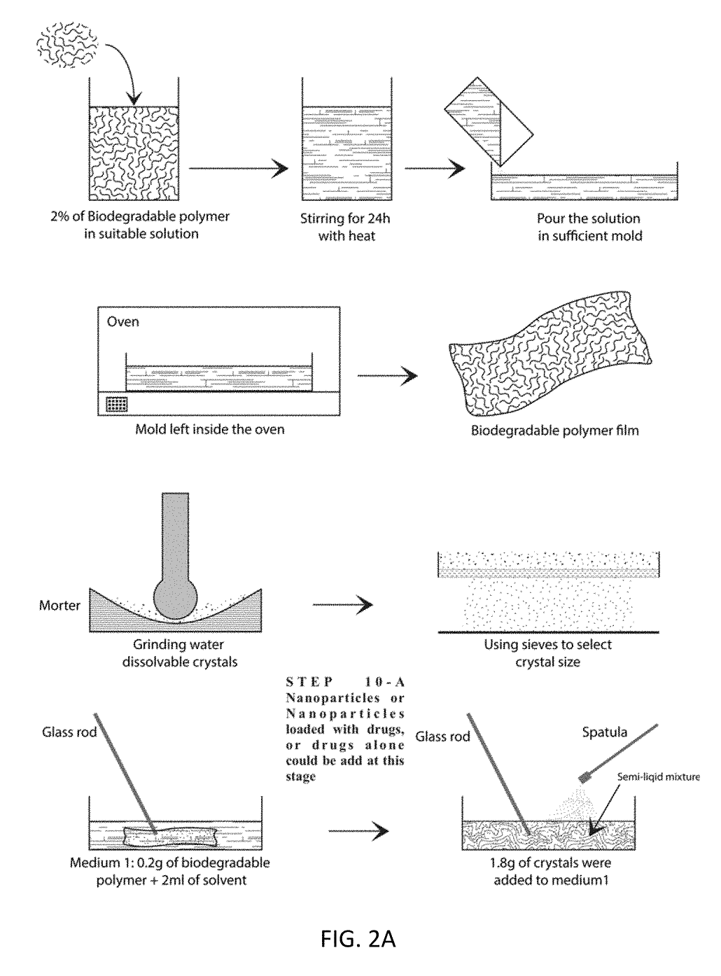

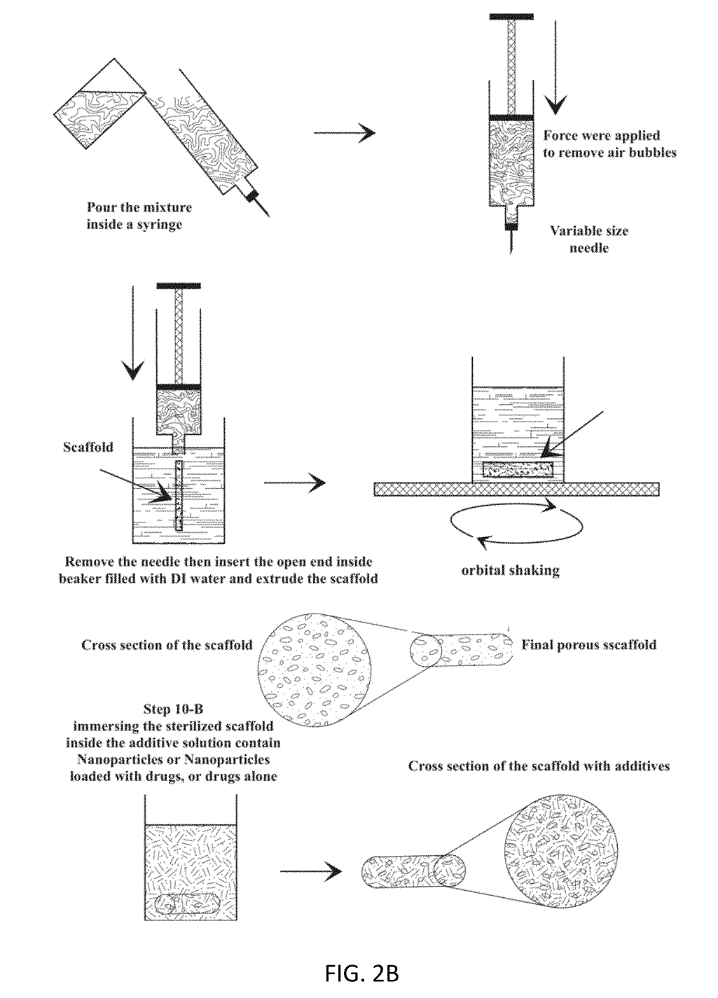

FIGS. 2A and 2B show the method (steps) for fabricating the 3D scaffold according to the exemplary embodiment of the disclosure.

(1). Dissolving 2 g of polyurethane in 100 ml of 90% ethanol/10% DI water, leaving the solution under heat (60° C.) and stirring at 360 rpm for 48 hours. In general, different biodegradable and biocompatible polymers can be used in place of polyurethane. The polymer amount used can also be changed through this step.

(2). Purging the mixture in a proper mold, and then keeping it inside a preheat oven with a specific temperature overnight.

(3). Grinding the soluble crystals by using the mortar and pestle, then selecting specific crystal size by using the sieves with different mesh size, for example crystals range from 75 to 150 μm. Crystal size can be altered depending on the design.

(4). To prepare medium A, cutting 0.2 g of previously prepared thin film and putting it inside the mortar, then adding 2 ml of absolute ethanol, waiting for suitable time till the polymer become very soft and easy to mix. Generally, component for medium A can be changed, depending on the type of biodegradable polymer used.

(5). Adding 1.8 g of soluble crystals medium B with selective crystal size to the mortar, then mixing it with the medium A until a paste-like state is achieved. In general, the mixing ratio between biodegradable polymer and the soluble crystals can be altered depend on the quantity of the porosity within the scaffold, in this mixture case around 90% porosity were achieved within the structure.

(6). Placing the mixture inside a syringe with desired size and diameter. Applying pressure by a syringe plunger to the mixture, in order to remove any bubbles. Generally, different tools can be used to shape the composite in various shapes.

(7). Extruding the mixture by using the syringe inside a beaker contain DI water medium D, where the polymer solidifies once it comes in contact with water and it take the shape and size of open end of the syringe. Generally, scaffold dimension range from 0.5 nm to 30 cm. DI water can be altered with other liquids.

(8). Gently transferring the scaffold in a water bath that is placed on an orbital shaker. Keeping the scaffold for suitable period inside the water bath under orbital shake to allow leaching of the soluble crystals with DI water; exchanging DI water once every 10 to 12 hours, this process is continues till the soluble crystals are totally removed from the structure.

(9). After a complete leaching of solvent dissolvable crystals, placing the scaffold under vacuum until completely dry. Sterilization is accomplished by washing it twice with 1× PBS and DI water followed by exposure to UV light overnight.

(10). Incubating or incorporating nanoparticles and tissue regeneration enhancing drugs within the scaffold. The step can be performed as follows:

(A) Direct addition of the nanoparticles, microparticles, nanoparticles or microparticles loaded with drugs, or drugs alone within the mixture prepare by step (5), followed by next normal steps.

(B) Loading the nanoparticles, microparticles which can be loaded with drugs, cells, etc. within the scaffold is accomplished by immersing the sterilized scaffold inside the solution contain nanoparticles or nanoparticles loaded with drugs, or drugs alone cells, etc., for a specific period.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments are chosen and described in order to explain the principles of the disclosure and their practical application so as to activate others skilled in the art to utilize the disclosure and various embodiments and with various modifications as are suited to the particular use contemplated. Alternative embodiments will become apparent to those skilled in the art to which the present disclosure pertains without departing from its spirit and scope. Accordingly, the scope of the present disclosure is defined by the appended claims rather than the foregoing description and the exemplary embodiments described therein.

### REFERENCE LIST

[1]. ALGHAZALI, K. M., NIMA, Z. A., HAMZAH, R. N., DHAR, M. S., ANDERSON, D. E. and BIRIS, A. S. 2015. Bone-tissue engineering: complex tunable structural and biological responses to injury, drug delivery, and cell-based therapies. Drug Metabolism Reviews, 47, 431-454.
[2]. D O, A.-V., KHORSAND, B., GEARY, S. M. & SALEM, A. K. 2015. 3D Printing of Scaffolds for Tissue Regeneration Applications. Advanced healthcare materials, 4, 1742-1762.
[3]. IKADA, Y. 2006. Challenges in tissue engineering. Journal of the Royal Society Interface, 3, 589-601.
[4]. KEATING, J. F. and MCQUEEN, M. M. 2001. Substitutes for autologous bone graft in orthopaedic trauma. J Bone Joint Surg Br, 83, 3-8.
[5]. PANGARKAR, N. and HUTMACHER, D. W. 2003. Invention and business performance in the tissue-engineering industry. Tissue Eng, 9, 1313-22.
[6]. ROUSSEAU, M., ANDERSON, D. E., LILLICH, J. D., APLEY, M. D., JENSEN, P. J. and BIRIS, A. S. 2014. In vivo assessment of a multicomponent and nanostructural polymeric matrix as a delivery system for antimicrobials and bone morphogenetic protein-2 in a unicortical tibial defect in goats. Am J Vet Res, 75, 240-50.

What is claimed is:

1. A scaffold useable for tissue regeneration, comprising:
a three-dimensional (3D) structure having a tunable porosity with interconnected channels and pores along with adjustable dimensions, and being formed of at least one of a first medium, a second medium, a third medium and a fourth medium,
wherein the first medium comprises one or more polymers that are biocompatible and biodegradable;
wherein the second medium comprises one or more soluble materials, and is mixable with the first medium;
wherein the third medium comprises fillers of one or more insoluble materials having structures with dimensions between 1 nm to 5 mm, and is mixable in a bulk or surface of the first medium or the second medium individually, or in a bulk or surface of a combination of the first and second media; and
wherein the fourth medium comprises an agent.

2. The scaffold of claim 1, wherein the 3D structure is capable of incubating or incorporating various types of nanoparticles, cells, bioactive materials, growth factors, and/or tissue regeneration enhancing drugs therein.

3. The scaffold of claim 1, wherein internal and external surfaces of the 3D structure and/or a bulk of the 3D structure are coated with nanostructural materials.

4. The scaffold of claim 1, wherein the 3D structure has a shape and size conforming to a shape and size of corresponding tissue that needs to be regenerated.

5. The scaffold of claim 1, wherein a mixture of the first, second and third media is obtained in bulks, layers, or concentrically arranged geometries by using at least one process of mixing, spraying, electrospraying, extrusion, layer-by-layer deposition, and the likes.

6. The scaffold of claim 5, wherein the mixture of the first, second and third media is operably exposed to the fourth medium to remove the second medium without adversely affecting the first and third media, so as to form a first composite.

7. The scaffold of claim 6, wherein the fourth medium is operably removed from the first composite by at least one process of evaporating, drying, heating, vacuum drying, freeze-drying, and the likes, so as to form a second composite.

8. The scaffold of claim 7, wherein the second composite is operably exposed to a plasma treatment for the surface modification to alter its surface chemistry, wherein the plasma treatment is performed in at least one gas of oxygen, nitrogen, helium, argon, and the likes.

9. The scaffold of claim 7, wherein a concentration of the third medium is between 0 to 99.99% of the first medium in the second composite.

10. The scaffold of claim 1, wherein the tunable porosity of the 3D structure is tunable with pore sizes from 0.1 nm to 10 mm, and wherein the surface area of the 3D structure is between 0.001 and 5000 m²/g.

11. The scaffold of claim 1, wherein the tunable porosity is achievable through 3D printing.

12. The scaffold of claim 1, wherein the one or more polymers comprise polyurethanes, polylactide (PLA), polyglycolide (PGA), poly(lactide-co-glycolide) (PLGA), poly(e-caprolactone), polydioxanone, polyanhydride, trimethylene carbonate, poly(β-hydroxybutyrate), poly(g-ethyl glutamate), poly(desaminotyrosinetyrosylhexyl ester iminocarbonate) (poly(DTH iminocarbonate)), poly(bisphenol A iminocarbonate), poly(ortho ester), polycyanoacrylate, polyphosphazene, a polymer derived from natural source including polysaccharides, proteins, or a mixture thereof.

13. The scaffold of claim 1, wherein the first medium is combinable with ethanol, methanol, or other organic solvents or mixtures thereof.

14. The scaffold of claim 1, wherein the one or more soluble materials have a rate of degradation or dissolution that is faster than that of the first medium in a solvent, and comprise soluble crystals including sodium, chloride, sugar, or other material.

15. The scaffold of claim 1, wherein the one or more insoluble materials comprise at least one of
(1) metal materials including gold, silver, copper, or other metals, or a combination of them, with micro-sized and/or nano-sized structures of various shapes including spheres, rods, platelets, cylinders, cubes, pyramids, cavities, nanoshells, nanocages, or the likes;
(2) carbonaceous materials including nanotubes, graphene, nanofibers, nanoonions, nanocones, or the likes;
(3) micro-sized or nano-sized hydroxyapatite;
(4) bone component particles, and/or bone component nanoparticles;
(5) calcium phosphate; or
(6) micro-sized and/or micro-sized ceramics.

16. The scaffold of claim 1, wherein the agent comprises deionized (DI) water, sodium hydroxide, ethanol, methanol, or other organic solvents, or mixtures thereof.

17. A method for fabricating a scaffold useable for tissue regeneration, comprising:
providing a first medium, a second medium, a third medium and a fourth medium, wherein the first medium comprises one or more polymers that are biocompatible and biodegradable; wherein the second medium comprises one or more soluble materials, and is mixable with the first medium; wherein the third medium comprises fillers of one or more insoluble materials having structures with dimensions between 1 nm to 10 mm, and is mixable in a bulk or surface of the first medium or the second medium individually, or in a bulk or surface of a combination of the first and second media; and wherein the fourth medium comprises an agent;
forming a mixture of the first, second and third media in bulks, layers, or concentrically arranged geometries by at least one process of mixing, spraying, electrospraying, extrusion, layer-by-layer deposition, and the likes;
exposing the mixture of the first, second and third media to the fourth medium to remove the second medium without adversely affecting the first and third media, so as to form a first composite; and
removing the fourth medium from the first composite by at least one process of evaporating, drying, heating, vacuum drying, freeze-drying, and the likes, so as to form the scaffold,
wherein the scaffold comprises a three-dimensional (3D) structure having a tunable porosity with interconnected channels and pores along with adjustable dimensions.

18. The method of claim 17, further comprising performing a plasma treatment to the scaffold for the surface modification to alter its surface chemistry, wherein the plasma treatment is performed in at least one gas of oxygen, nitrogen, helium, argon, and the likes.

19. The method of claim 17, wherein the 3D structure is capable of incubating or incorporating various types of nanoparticles, cells, bioactive materials, growth factors, and/or tissue regeneration enhancing drugs therein.

20. The method of claim 17, wherein internal and external surfaces of the 3D structure and/or a bulk of the 3D structure are coated with nanostructural materials.

21. The method of claim 17, wherein the 3D structure has a shape and size conforming to a shape and size of corresponding tissue that needs to be regenerated.

22. The method of claim 17, wherein a concentration of the third medium is between 0 to 99.99% of the first medium in the scaffold.

23. The method of claim 17, wherein the tunable porosity of the 3D structure is tunable with pore sizes from 0.1 nm to 10 mm, and wherein the surface area of the 3D structure is between 0.001 and 5000 m²/g.

24. The method of claim 17, wherein the tunable porosity is achievable through 3D printing.

25. The method of claim 17, wherein the one or more polymers comprise polyurethanes, polylactide (PLA), polyglycolide (PGA), poly(lactide-co-glycolide) (PLGA), poly(e-caprolactone), polydioxanone, polyanhydride, trimethylene carbonate, poly(β-hydroxybutyrate), poly(g-ethyl glutamate), poly(desaminotyrosinetyrosylhexyl ester iminocarbonate) (poly(DTH iminocarbonate)), poly(bisphenol A iminocarbonate), poly(ortho ester), polycyanoacrylate, polyphosphazene, a polymer derived from natural source including polysaccharides, proteins, or a mixture thereof.

26. The method of claim 17, wherein the first medium is combinable with ethanol, methanol, or other organic solvents or mixtures thereof.

27. The s method of claim 17, wherein the one or more soluble materials have a rate of degradation or dissolution that is faster than that of the first medium in a solvent, and comprise soluble crystals including sodium, chloride, sugar, or other material.

28. The method of claim 17, wherein the one or more insoluble materials comprise at least one of
(1) metal materials including gold, silver, copper, or other metals, or a combination of them, with micro-sized and/or nano-sized structures of various shapes including spheres, rods, platelets, cylinders, cubes, pyramids, cavities, nanoshells, nanocages, or the likes;
(2) carbonaceous materials including nanotubes, graphene, nanofibers, nanoonions, nanocones, or the likes;
(3) micro-sized or nano-sized hydroxyapatite;
(4) bone component particles, and/or bone component nanoparticles;
(5) calcium phosphate; or
(6) micro-sized and/or micro-sized ceramics.

29. The method of claim 17, wherein the agent comprises of deionized (DI) water, sodium hydroxide, ethanol, methanol, or other organic solvents, or mixtures thereof.

30. A method for fabricating a scaffold useable for tissue regeneration, the scaffold having a three-dimensional (3D) structure having a tunable porosity with interconnected channels and pores along with adjustable dimensions, comprising:
providing a first medium, a second medium, a third medium and a fourth medium, wherein the first medium comprises one or more polymers that are biocompatible and biodegradable; wherein the second medium comprises one or more soluble materials, and is mixable with the first medium; wherein the third medium comprises fillers of one or more insoluble materials having structures with dimensions between 1 nm to 5 mm, and is mixable in a bulk or surface of the first medium or the second medium individually, or in a bulk or surface of a combination of the first and second media; and wherein the fourth medium comprises an agent;
mixing the second medium with the first medium until a mixture-like state is achieved, to form a mixture, wherein the mixing ratio between biodegradable polymer and the soluble crystals could be altered depend on the quantity of the porosity within the scaffold, in this mixture case around 90% porosity were achieved within the structure;
exposing the mixture to the fourth medium to solidify the one or more polymers so as to form the scaffold;
transferring the scaffold in a water bath, or the third medium, that is placed on an orbital shaker and leaching the one or more soluble materials from the scaffold with DI water; and
drying and sterilizing the scaffold.

31. The method of claim 30, wherein the mixing step comprises adding nanoparticles microparticles, growth factors, and/or tissue regeneration enhancing drugs when mixing the first and second media to form the mixture, so that the nanoparticles microparticles, and/or tissue regeneration enhancing drugs are incubated and incorporated within the scaffold.

32. The method of claim 30, further comprising immersing the sterilized scaffold the inside the solution contain nanoparticles microparticles, growth factors, and/or tissue regeneration enhancing drugs for a predetermined period.

33. The method of claim 30, wherein the exposing step comprises placing the mixture in a syringe having desired size and diameter; and extruding the mixture by the syringe inside a container contains the fourth medium, so that the scaffold has a shape and size conforming to a shape and size of corresponding tissue that needs to be regenerated.

34. The method of claim 30, wherein the one or more polymers comprise polyurethanes, polylactide (PLA), polyglycolide (PGA), poly(lactide-co-glycolide) (PLGA), poly(e-caprolactone), polydioxanone, polyanhydride, trimethylene carbonate, poly(β-hydroxybutyrate), poly(g-ethyl glutamate), poly(desaminotyrosinetyrosylhexyl ester iminocarbonate) (poly(DTH iminocarbonate)), poly(bisphenol A iminocarbonate), poly(ortho ester), polycyanoacrylate, polyphosphazene, a polymer derived from natural source including polysaccharides, proteins, or a mixture thereof.

35. The method of claim 30, wherein the first medium is combinable with ethanol, methanol, or other organic solvents or mixtures thereof

36. The s method of claim 30, wherein the one or more soluble materials have a rate of degradation or dissolution that is faster than that of the first medium in a solvent, and comprise soluble crystals including sodium, chloride, sugar, or other material.

37. The method of claim 30, wherein the one or more insoluble materials comprise at least one of
(1) metal materials including gold, silver, copper, or other metals, or a combination of them, with micro-sized and/or nano-sized structures of various shapes including spheres, rods, platelets, cylinders, cubes, pyramids, cavities, nanoshells, nanocages, or the likes;
(2) carbonaceous materials including nanotubes, graphene, nanofibers, nanoonions, nanocones, or the likes;
(3) micro-sized or nano-sized hydroxyapatite;
(4) bone component particles, and/or bone component nanoparticles;
(5) calcium phosphate; or
(6) micro-sized and/or micro-sized ceramics.

38. The method of claim 30, wherein the agent comprises deionized (DI) water, sodium hydroxide, ethanol, methanol, or other organic solvents, or mixtures thereof.
